(19) United States
(12) Patent Application Publication
(10) Pub. No.: US 2011/0066117 A1
(43) Pub. Date: Mar. 17, 2011

(54) LARGE VOLUME ENEMA

(76) Inventors: Sarah S. Post, Forest, VA (US); Michael L. Caswell, Forest, VA (US); Charles H. Cox, Concord, VA (US)

(21) Appl. No.: 12/953,351

(22) Filed: Nov. 23, 2010

Related U.S. Application Data
(63) Continuation of application No. 10/846,488, filed on May 13, 2004, now abandoned.

(51) Int. Cl. A61M 3/02; A61K 33/42; A61P 1/00

(52) U.S. Cl. 604/257; 424/606

(57) ABSTRACT

An enema comprises a bottle, and at least 150 ml of a solution in the bottle. The solution comprises water, 5.00 to 8.00 grams dibasic sodium phosphate, and 15.00 to 21.00 grams monobasic sodium phosphate.

### BACKGROUND

Colonic purgative procedures typically involve the emptying of the colon using water based enemas: a solution is introduced into the rectum and colon to induce emptying. It has also been recognized that the introduction of a hypertonic aqueous solution, typically of various salts, is superior to a pure water enema. These salt solutions increase the osmotic pressure in the colon, drawing fluid into the colon and promoting bowel movement.

This type of enema, however, may have undesirable side effects, if the hypertonic solution diffuses through the wall of the colon and disturbs the fluid balance of the body. The United States Food and Drug Administration (USFDA) has therefore imposed a limit on the amount salts contained in an enema. For example, in enema formulations containing phosphate salts, the single daily dosage for adults or children 12 years of age and over is 6.84 to 7.56 grams of dibasic sodium phosphate (Na₂HPO₄) and 18.24 to 20.16 grams of monobasic sodium phosphate (NaH₂PO₄). See 21 CFR §334.58(5)(ii).

This limit on the amount of phosphates for a single daily dosage limits the volume of traditional phosphate-based enemas to about 133 ml (this volume corresponds with the volume in the bottle; the actual delivered dose is somewhat smaller), because lower concentrations of phosphates are believed to yield compositions too dilute to have sufficient osmotic pressure to provide the advantages associated with phosphate solution enemas.

Outside the United States, larger volume enemas are known. For example, in Spain a double size (266 ml) enema is available. The concentrations of the phosphate salts is the same as for enemas available in the United States, and therefore the dosage of phosphates is about double the limit set by the USFDA.

### BRIEF SUMMARY

In a first aspect, the present invention is an enema, comprising (a) a bottle, and (b) at least 150 ml of a solution in the bottle. The solution comprises (i) water, (ii) 5.00 to 8.00 grams dibasic sodium phosphate, and (iii) 15.00 to 21.00 grams monobasic sodium phosphate.

In a second aspect, the present invention is a method of bowel purging, comprising administering at least 150 ml of a solution rectally. The solution comprises (i) water, (ii) 5.00 to 7.56 grams dibasic sodium phosphate, and (iii) 15.00 to 20.16 grams monobasic sodium phosphate.

In a third aspect, the present invention is an enema solution, comprising (i) water, (ii) dibasic sodium phosphate in an amount of 0.0142 to 0.0504 grams/ml water, and (iii) monobasic sodium phosphate in an amount of 0.0428 to 0.1344 grams/ml water.

In a fourth aspect, the present invention is an enema bottle, comprising (1) a bottle having a commercially usable volume of 150 to 260 ml, and (2) a rectal administration tip, attached to the bottle.

### BRIEF DESCRIPTION OF THE FIGURES

FIG. 1 is a schematic of an enema bottle.

FIG. 2 is a graph of unpriced purchase intent of test respondents.

FIG. 3 is a graph of overall satisfaction of test respondents.

FIG. 4 is a graph of reasons for enema usage of test respondents.

FIG. 5 is a graph of the rating of satisfying intended purpose of test respondents.

FIG. 6 is a graph of product ratings of test respondents.

FIG. 7 is a graph of comparison ratings of test respondents.

FIG. 8 is a graph of likelihood of switching of test respondents.

FIG. 9 is a graph of satisfaction with enemas typically used of test respondents.

FIG. 10 illustrates a tip for an enema bottle.

FIG. 11 illustrates a protective shield for a tip of an enema bottle.

### DETAILED DESCRIPTION

The present invention makes use of the discovery that increasing the delivered volume of the enema to at least 150 ml, while maintaining the total amount of phosphate salts, yields an enema that is superior to smaller volume (133 ml) formulations containing similar total amounts of salts. This result is in contrast with the previously held belief that phosphate-based enemas with a volume over 133 mL would be too diluted to be effective, unless the amount of phosphates exceeded the USFDA limit.

The present invention includes a large volume enema. The enema contains at least 5.00 grams dibasic sodium phosphate, and at least 15 grams monobasic sodium phosphate. Preferably, the amount of dibasic sodium phosphate is 6.00 to 8.00 grams, more preferably 6.84 to 7.56 grams. Preferably, the amount of monobasic sodium phosphate is 17.00 to 21.00 grams, more preferably 18.00 to 20.16 grams, most preferably 18.24 to 20.16 grams. The volume of solution in the enema is at least 150 ml, preferably 180 ml to 350 ml, more preferably 200 ml to 300 ml, most preferably 210 ml to 260 ml.

These solutions may be prepared by dissolving the phosphate salts in water, reacting dibasic sodium phosphate with phosphoric acid, or reacting sodium hydroxide with phosphoric acid. The concentration of dibasic sodium phosphate is 0.0142 g/ml water to 0.0504 g/ml water, including 0.015, 0.020, 0.025, 0.030, 0.035, 0.040, 0.045 and 0.050 g/ml water. The concentration of monobasic sodium phosphate is 0.0428 g/ml water to 0.1344 g/ml water, including 0.045, 0.050, 0.060, 0.070, 0.080, 0.090, 0.100, 0.110, 0.120, and 0.130 g/ml water.

Optionally, the enema may also contain additives. For instance surfactants, preservatives such as benzalkonium chlorides (alkylbenzyldimethylammonium chlorides) and EDTA, and fragrances may be added.

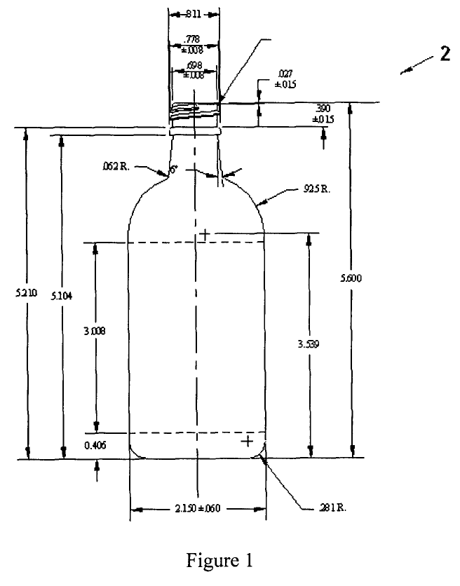

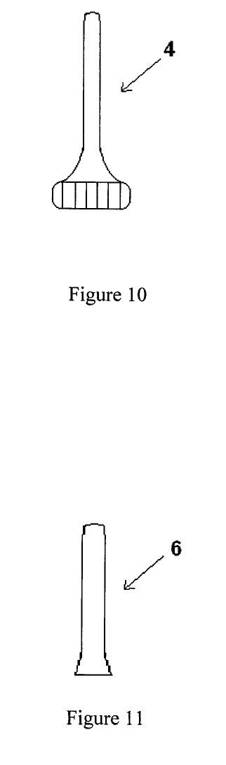

The large volume enema is provided in an enema bottle 2 (for example, the bottle illustrated in FIG. 1; the lengths listed are in inches). The bottle is attached to a rectal administration tip 4 (for example, the tip illustrated in FIG. 10), that is preferably lubricated. A protective shield 6 (for example, the shield illustrated in FIG. 11) helps maintain the lubricant in place on the tip during storage. Preferably, the bottle is composed of a soft plastic that is squeezable, allowing delivery of the enema solution after the tip is place through the anus. The tip contains a valve, preferably of a soft rubber disk which contains a slit that acts as a one-way valve, preventing fluid for accidentally returning into the bottle during use. The entire bottle is disposable after use, and is preferably not intended for re-use.

The specification of the enema bottle illustrated in FIG. 1 provide for a volume of solution of 230.88 ml, since the bottle may only be filled to the shoulder for commercial use. The maximum volume to which a bottle may be filled for commercial use is referred to as the commercially usable volume. The bottle may have commercially usable volume of at least 150 ml, preferably 180 to 350 ml, more preferably 200 to 300 ml, most preferably 210 to 260 ml, including 220, 230, 240 and 250 ml. The volume actually delivered from the 230.88 ml bottle, on average, is about 193 ml, with a minimum amount delivered of about 180 ml; this was determined by test squeezing 50 bottles, using an unlubricated tip, and measuring the amount of liquid expelled from the bottle.

In the example below, it was unexpectedly discovered that an enema volume of about 230 ml (with an average delivered volume of about 193 ml, and an expected minimum delivered amount during normal use of 180 ml), but with the total amount of phosphates unchanged from a typical enema (133 ml enema volume) resulted in significantly higher consumer approval. The data below demonstrates that this large volume enema proved superior to the typical volume enema for a variety of commercially significant criteria.

### Example

Enemas containing 19 g of monobasic sodium phosphate (NaH₂PO₄) and 7 g of dibasic sodium phosphate (Na₂HPO₄) dissolved in 230.88 ml of water were packed in the bottles. The large volume enema was compared with normal volume enemas in a study of typical enema users. 149 respondents were mailed product to get approximately 100 completed interviews. Home-use test respondents were mailed a box via U.S. Priority mail containing two 7.8 fl. oz. enemas (unbranded), a sheet of instructions, and a questionnaire to record their responses before completing the online survey. After using the product and completing the paper questionnaire, respondents went to a website to record their responses online and to answer some additional questions. Respondents for the home-use test were people ages 18-64 who use a store-purchased enema at least twice a month.

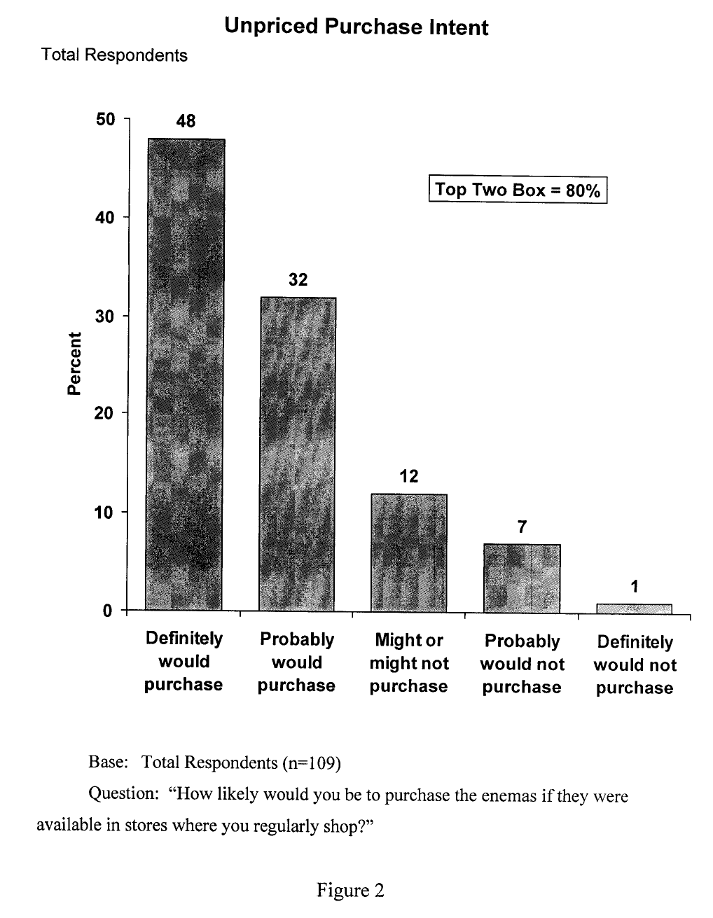

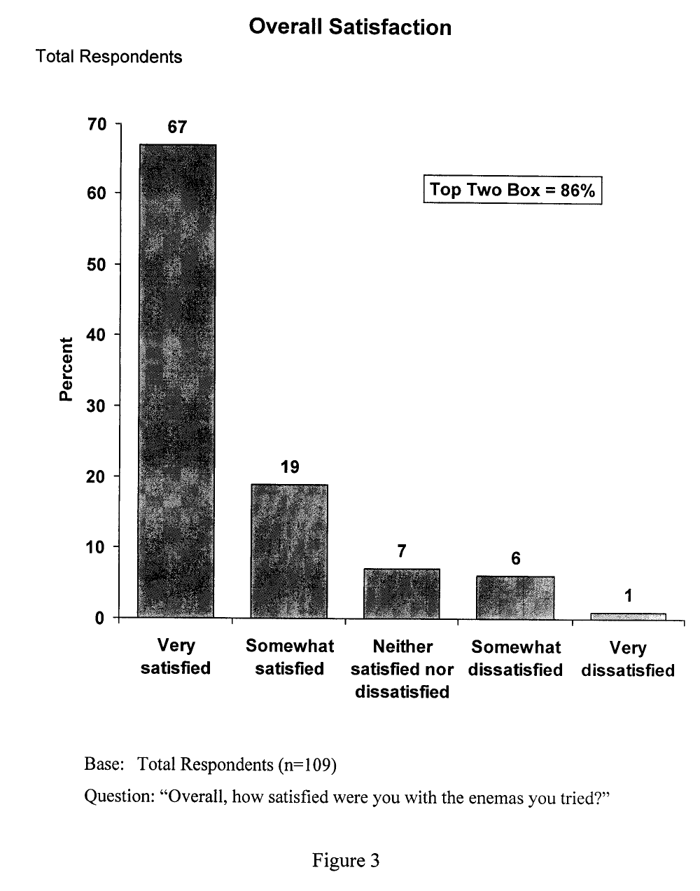

Overall, reactions to the large volume enemas were positive. Unpriced purchase intent was high, with 80% of enema users saying they “definitely” or “probably would purchase,” and overall satisfaction was even higher (86% “very” or “somewhat satisfied”). See FIGS. 2 and 3.

When asked what they like about the large volume enema they tried, the most frequently mentioned comments were related to how well the enema worked, the ease of use and comfort. Though nearly half of enema users (47%) made positive comments about the enema's performance, one out of ten (10%) gave answers related to performance when asked what they disliked about the large volume enema. In comparison, only 40% made positive comments about the enema's performance, and 26% gave answers related to performance, when asked what they disliked about the enema they typically used. See the tables 1-4 below.

TABLE 1
Likes

| | Total |
|---|---|
| Base = Total Respondents | (109) |
| Works Well/Good Performance (Net) | 47% |
| Works quickly | 18 |
| Works/works well | 12 |
| Effective | 10 |
| Effective/performance | 5 |
| Cleanses well | 5 |
| Relieves constipation | 5 |
| Ease Of Use (Net) | 28% |
| Easy to use/easy application | 25 |
| Easy to squeeze | 6 |
| Larger size/more solution | 15 |
| Comfort/No Irritation (Net) | 15% |
| Comfortable | 6 |
| No cramping | 4 |
| Gentle to use/gentle | 3 |
| Lubricated top/tip/lubrication | 6 |
| Size of bottle/right amount of solution | 6 |
| Smooth | 3 |
| Soft/soft tip | 3 |
| Other | 17 |
| Nothing | 6 |

Question: “What, if anything, did you like about the enemas you tried?”

TABLE 2
Dislikes

| | Total |
|---|---|
| Base = Total Respondents | (109) |
| Does Not Work Well (Net) | 10% |
| Does not work well | 5 |
| Does not work as fast | 3 |
| Does not clean me out completely | 3 |
| Bottle (Net) | 8% |
| Bottle too large/too much liquid | 6 |
| Applicator | 7 |
| Irritation (Net) | 7% |
| Stinging sensation/burning/discomfort | 4 |
| Hard to use | 5 |
| Other | 12 |
| Nothing | 55 |

Question: “What, if anything, did you dislike about the enemas you tried?”

TABLE 3
Likes - Enema Typically Used

| | Total |
|---|---|
| Base = Total Respondents | (109) |
| Works Well/Good Performance (Net) | 40% |
| Works/works well | 15 |
| Effective | 9 |
| Works quickly | 8 |
| Cleanses well | 5 |
| Relieves constipation | 5 |
| Price | 20 |
| Ease Of Use (Net) | 13% |
| Easy to use/easy application | 11 |
| Availability/easy to find in stores | 7 |
| Convenience | 6 |
| Comfort/No Irritation (Net) | 6% |
| No cramping | 3 |
| Used to the product | 4 |
| Lubricated top/tip/lubrication | 3 |
| Other | 17 |
| Nothing | 13 |

Question: “What, if anything, do you like about the enema you typically use?”

TABLE 4
Dislikes - Enema Typically Used

| | Total |
|---|---|
| Base = Total Respondents | (109) |
| Bottle (Net) | 26% |
| Bottle too small/need more than one | 17 |
| bottle | |
| Difficult to squeeze bottle | 10 |
| Does Not Work Well (Net) | 13% |
| Does not work well | 5 |
| Does not clean me out completely | 5 |
| Applicator (Net) | 6% |
| Applicator does not have enough | 3 |
| lubricant | |
| Irritation (Net) | 6% |
| Stinging sensation/burning/discomfort | 3 |
| Makes me cramp | 3 |
| Hard to use | 5 |
| The mess/messy to apply | 3 |
| Other | 19 |
| Nothing | 37 |

Question: “What, if anything, do you dislike about the enema you typically use?”

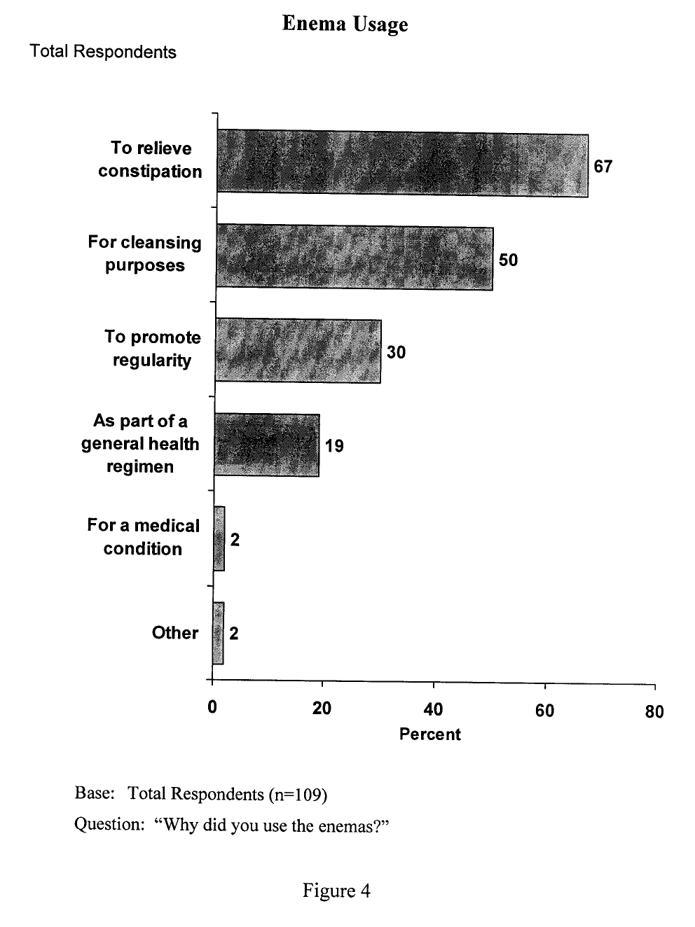

The most frequently mentioned reason for using the enemas was “to relieve constipation” (67%), followed by “for cleansing purposes” (50%) and “to promote regularity” (30%). Over half of the enema users (56%) rated the enema as “excellent” in satisfying the intended purpose. The test enema was rated better than the enema typically used on the following attributes: “overall performance” (64%), “relieves constipation” (63%), “cleanses well” (57%), and “easy to squeeze” (50%). See Figure 4. Nearly all enema users (more than 90%) found the test enema to be about the same or better than the enema they typically use on any attribute that they rated.

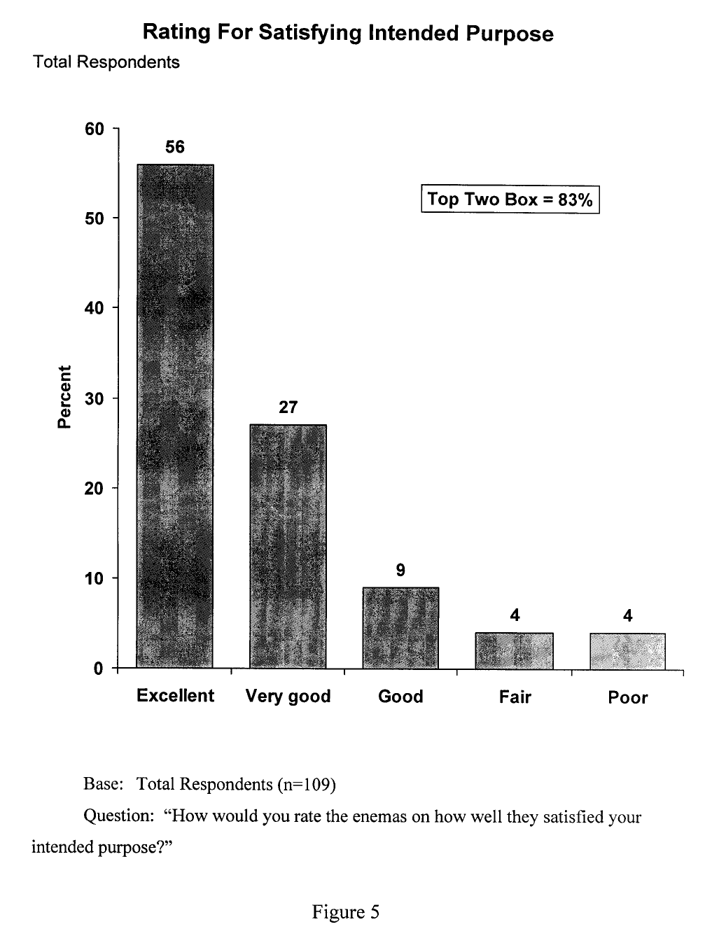

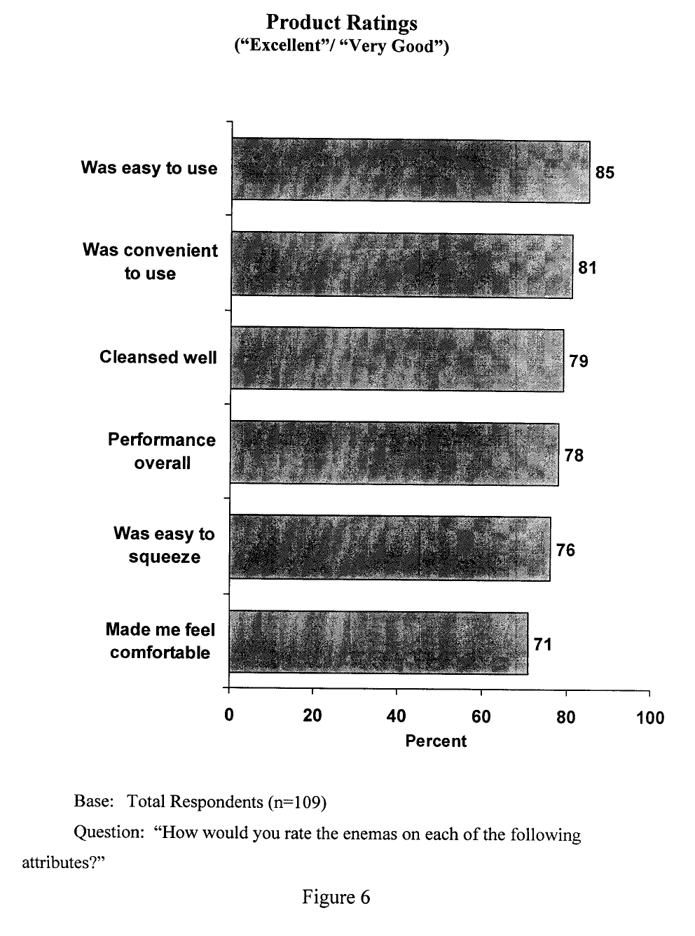

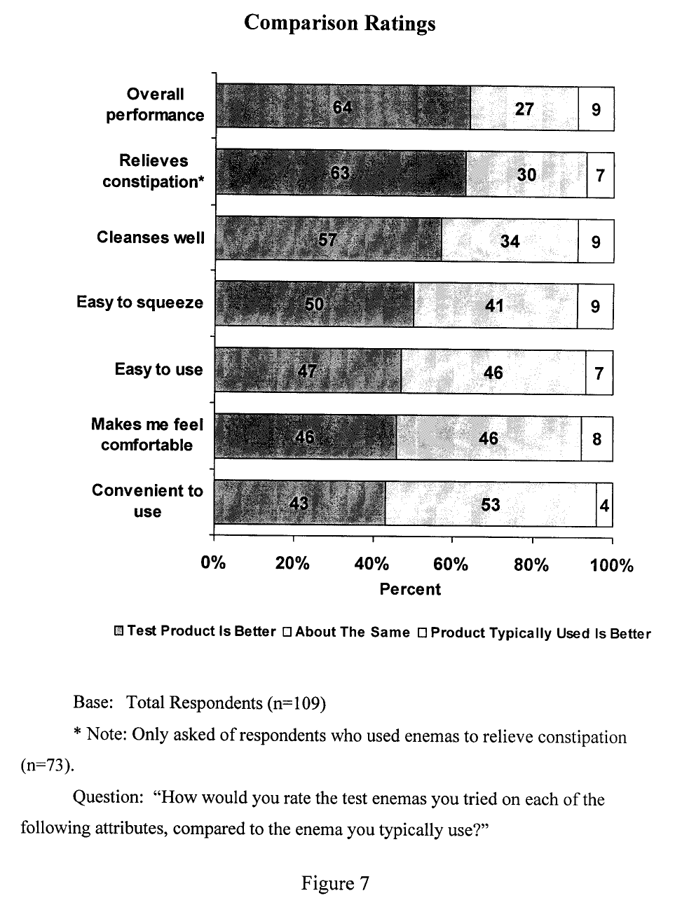

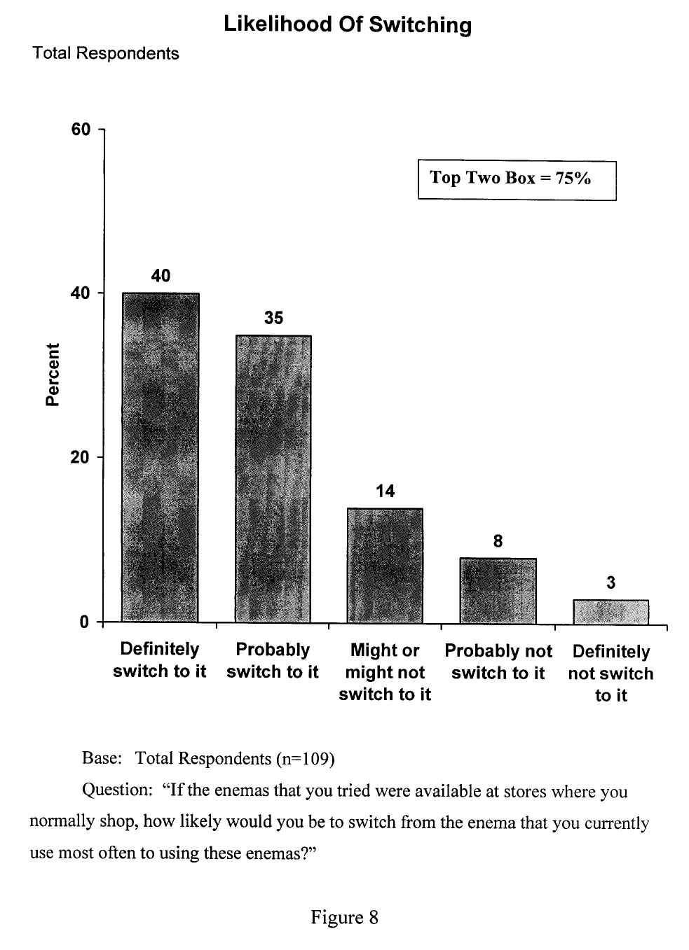

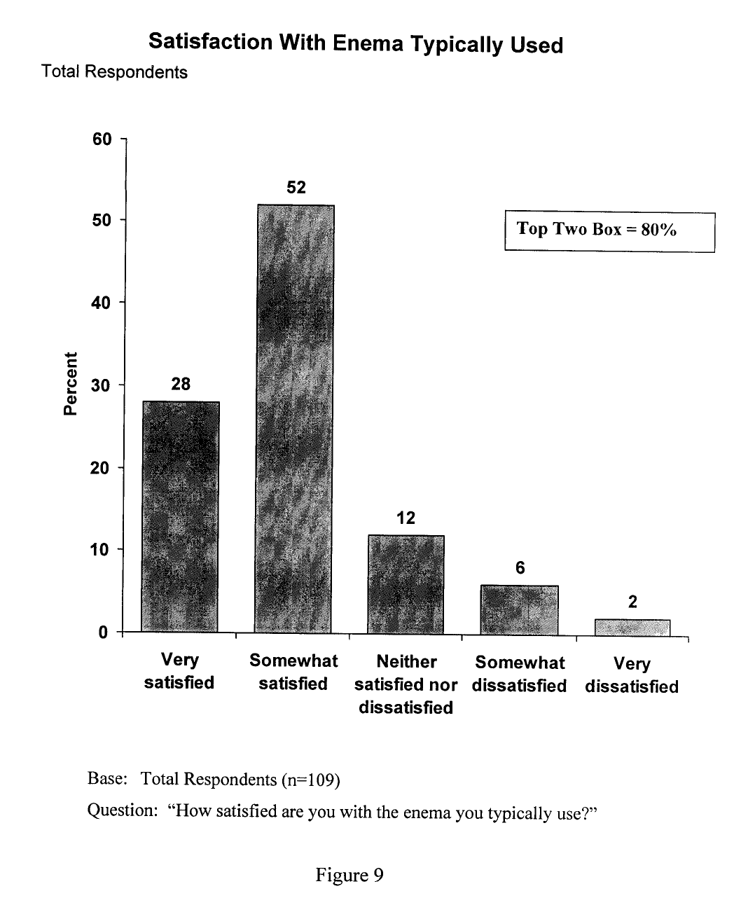

When asked if they would switch, three out of four enema users say they would switch from the enema they typically use to the enema they tested. The primary reason for switching was that the larger size enema worked better. Other reasons mentioned by those that said they would switch were related to the bottle and comfort. Price plays a part in determining whether or not enema users would switch. One third of enema users who said they might switch or would not switch mentioned price as a reason. However, enema users indicated that they would be willing to pay more for the enema they tried in the product test than what they currently pay for enemas they typically use. See table 5 below. FIGS. 5-9 provide additional data demonstrating the superior performance of the large volume enema.

TABLE 5
Reasons for Switching

| | Total |
|---|---|
| Base = Definitely or probably switch to it | (82) |
| Works Well/Good Performance (Net) | 39% |
| Worked well/worked better | 26 |
| Cleanses better | 15 |
| Worked faster | 5 |
| Like it/like it better | 17% |
| Bottle (Net) | 16% |
| Larger size/bottle | 11 |
| Bottle is easier to handle/squeeze | 7 |
| Comfort/No Irritation (Net) | 15% |
| Comfortable/comfortable to use | 9 |
| All other comfort mentions | 4 |
| Easy to use | 13 |
| Good/better product | 9 |
| More fluid/one bottle has enough fluid | 9 |
| Price (Net) | 5% |
| Good price | 5 |
| Convenience | 4 |
| Other | 12 |

Question: “Why would you definitely or probably switch to it?”

1. An enema, comprising:
(a) a bottle, and
(b) at least 150 ml of a solution in the bottle, comprising
(i) water,
(ii) 5.00 to 8.00 grams dibasic sodium phosphate, and
(iii) 15.00 to 21.00 grams monobasic sodium phosphate.

2. The enema of claim 1, wherein the bottle contains 180 to 350 ml of the solution.

3. The enema of claim 1, wherein the bottle contains 200 to 300 ml of the solution.

4. The enema of claim 1, wherein the bottle contains 210 to 260 ml of the solution.

5. The enema of claim 1, wherein the solution contains 6.00 to 8.00 grams dibasic sodium phosphate.

6. The enema of claim 1, wherein the solution contains 6.84 to 7.56 grams dibasic sodium phosphate.

7. The enema of claim 1, wherein the solution contains 17.00 to 21.00 grams monobasic sodium phosphate.

8. The enema of claim 1, wherein the solution contains 18.24 to 20.16 grams monobasic sodium phosphate.

9. The enema of claim 1, further comprising (c) a rectal administration tip, attached to the bottle.

10. The enema of claim 9, further comprising (d) a protective shield, on the tip.

11. The enema of claim 9, further comprising (e) lubricant, on the tip.

12. The enema of claim 1, wherein
the bottle contains 210 to 260 ml of the solution, and
the solution contains 6.84 to 7.56 grams dibasic sodium phosphate and 18.24 to 20.16 grams monobasic sodium phosphate.

13. The enema of claim 12, further comprising:
(c) a rectal administration tip, attached to the bottle,
(d) a protective shield, on the tip, and
(e) lubricant, on the tip.

14. A method of bowel purging, comprising:
administering at least 150 ml of a solution rectally;
wherein the solution comprises
(i) water,
(ii) 5.00 to 7.56 grams dibasic sodium phosphate, and
(iii) 15.00 to 20.16 grams monobasic sodium phosphate.

15. The method of claim 14, wherein the administering is administering at least 180 ml of the solution rectally.

16. The method of claim 14, wherein the administering is administering 180 to 300 ml of the solution rectally.

17. The method of claim 14, wherein the administering is administering 180 to 200 ml of the solution rectally.

18. The method of claim 14, wherein the solution comprises 6.84 to 7.56 grams dibasic sodium phosphate, and 18.24 to 20.16 grams monobasic sodium phosphate.

19. (canceled)

20. An enema solution, comprising:
(i) water,
(ii) dibasic sodium phosphate in an amount of 0.0142 to 0.0504 grams/ml water, and
(iii) monobasic sodium phosphate in an amount of 0.0428 to 0.1344 grams/ml water.

21-23. (canceled)

24. An enema bottle, comprising:
(1) a bottle having a commercially usable volume of 150 to 260 ml, and
(2) a rectal administration tip, attached to the bottle.

25-30. (canceled)
